(19) United States
(12) Patent Application Publication
(10) Pub. No.: US 2019/0374500 A1
(43) Pub. Date: Dec. 12, 2019

(54) Process for Preparing Water Soluble Azadiractin Powder Formulation with In-Built and External Inclusion Complexes

(71) Applicant: Triclad Biotech Private Ltd., Bangalore (IN)

(72) Inventors: Suvarna Shenvi, Bangalore (IN); Anil Kumar Kush, Bangalore (IN); G. Chandrasekara Reddy, Bangalore (IN)

(21) Appl. No.: 16/436,267

(22) Filed: Jun. 10, 2019

(30) Foreign Application Priority Data
Jun. 11, 2018 (IN) 201841021803

(51) Int. Cl. A61K 31/34; A61K 47/40; A61K 36/58; A61K 9/19

(52) U.S. Cl. CPC A61K 31/34; A61K 9/19; A61K 36/58; A61K 47/40

(57) ABSTRACT

Process for producing water soluble Azadirachtin containing powder formulation from neem seed kernel with the help of native and external inclusion complex. The process comprises expelling a large proportion of oil from neem seed kernels (NSK) by cold pressing, extracting the so called de-oiled neem seed cake with water to form an aqueous solution in which the azadirachtin is complexed with water soluble polysaccharides and proteins. The enrichment of azadiractin in aqueous solution is achieved by addition of azadiractin containing inclusion complex and spray drying. This helps in retaining all naturally occurring limonoids of neem seed kernel along with native encapsulating agents such as polysaccharide-protein matrix and also achieve water soluble powder with higher amounts of azadirachtin (>6000-60,000 ppm) in it.

### RELATED PATENT APPLICATION AND INCORPORATION BY REFERENCE

This is a utility application based upon and claims priority from patent application 201841021803 filed on Jun. 11, 2018 in India. This related application is incorporated herein by reference in its entirety and made a part of this application. If any conflict arises between the disclosure of the invention in this utility application and that in the related patent application, the disclosure in this utility application shall govern. Moreover, the inventor(s) incorporate herein by reference any and all patents, patent applications, and other documents hard copy or electronic, cited or referred to in this application.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### BACKGROUND OF THE INVENTION

### (1) Field of the Invention

The invention generally relates to a process of making high azadirachtin containing aqueous solution and then making it in powder form.

### (2) Description of the Related Art

The key insecticidal activity found in neem seeds is due to AZADIRACHTIN, a naturally occurring substance that belongs to an organic molecule called tetranortriterpenoid class. It is known that the seeds and other parts of the neem tree (Azadirachta indicia and related species) contain natural pesticidal compositions that cause feeding inhibition and growth inhibition in a variety of organisms including insects, mites and nematodes. Generally the term azadirachtin is taken to include all insecticidal terpenoids present in neem seed extract that partition along with azadirachtin.

In recovering azadirachtin from neem seeds it is necessary to separate oil content to the maximum extent before attempting to extract with water. Water soluble constituents may include sugars, proteins along with azadiractin. These separation procedures are complicated by the fact that azadirachtin is susceptible to hydrolysis in water and to heat degradation.

Conventional methods (U.S. Pat. Nos. 4,943,434, 5,281,618, 5,391,779, 5,397,571, 5,395,951, 5,695,763) for extracting azadirachtin from neem seeds involve the usage of three organic solvents and two liquid/liquid partition steps. Firstly, the neem seeds are pressed to remove the majority of the neem oil. The resulting expeller cake is then extracted with methanol. The methanol extracts a wide range of substances including azadirachtin and the other triterpenies, diterpenies, the residual oil, and some polysaccharides and proteins. To produce a powder from the methanolic supernatant, the extract undergoes a number of clean-up steps.

In the first clean-up step, the supernatant is concentrated and partitioned against hexane, or a similar non-polar solvent, to remove the oils and diterpenes. The hexane is then driven off in a still and the resulting oil is collected. The second clean-up step involves taking the de-oiled supernatant and driving off the methanol. The resulting tar is then dissolved in ethyl acetate, or a similar solvent, and partitioned against water to remove the polysaccharides and water soluble proteins. The ethyl acetate which contains the azadirachtin and other triterpenes is then evaporated to produce an azadirachtin rich powder.

There are a number of problems associated with these conventional methods, with the major problem being cross-contamination of the solvents used in the process and the resulting variation in product quality. While good quality azadirachtin powder can be produced when first using the process with fresh solvents, after a number of cycles, problems with cross-contamination do arise. While hexane and methanol are normally considered essentially immiscible, in a multi-component system, such as is created during the extraction of azadirachtin described above, the hexane and methanol do demonstrate some miscibility. The change in the polarity of the solvents allows oils to be carried through the hexane partition and creates difficulties in producing a non-oily or flowable powder. A more serious problem is the potential of cross-contamination between the methanol and ethyl acetate. If the methanol is not removed in the drying step after the hexane partition, cross-contamination with ethyl acetate occurs. This contamination of the ethyl acetate with methanol causes azadirachtin to be carried over into the water in the ethyl acetate/water partition or in the worst case prevents a partition forming at all.

A further problem with the conventional methods for extracting azadirachtin is the production of waste water with a high biological oxygen demand (BOD). In many countries, the release of waste water with high BOD is not permitted and requires the installation of a relatively expensive water treatment facility.

An alternative azadirachtin extraction process is described in Australian patent no 661482 to Trifolio-M GmbH, Herstellung Und Vertrieb Hochreinier Biosubstanzen. In this alternative process, the neem seed is pressed or crushed to remove the majority of the oil and the expeller cake is extracted with warm water. The warm water extraction removes the azadirachtin, some of the more polar triterpenes, the majority of the polysaccharides and water soluble proteins, and a slight amount of the more polar oily compounds. The aqueous supernatant is partitioned against a solvent of intermediate polarity, such as ethyl acetate or dichloromethane with the azadirachtin partitioning into the organic layer. The organic layer can then be concentrated under vacuum and the azadirachtin precipitated through the addition of a non-polar solvent, such as hexane or petroleum ether. While high yields of azadirachtin powder are produced using this alternative process, the process still results in the discharge of an aqueous solution loaded with polysaccharides and proteins which, in some countries, will require treatment to meet environmental standards. Further, the precipitation step where a non-polar solvent is added to the concentrated supernatant results in cross-contamination with its attendant problems and expense of requiring purification of the solvents prior to their re-use in the extraction process.

As per U.S. Pat. No. 6,312,738, a process of producing an azadiractin-rich aqueous solution is disclosed which comprises deoiling by pressing whole neem seeds to remove a first proportion of the oil content; extracting the de-oiled neem seeds with polar organic solvent in which the azadiractin is soluble but in which polysaccharides and proteins are not substantially soluble; partitioning the organic solvent against water to form an aqueous solution of azadiractin.

In U.S. Pat. No. 6,340,484 the addition inert carriers such as talc, stealite, soap stone, pyrophyllite and yellow talc, kaolin and related carriers into aqueous neem extracts is described. But these are unsuitable for azadirachtin based formulations as they may degrade azadirachtin because of their acidity or alkalinity. More particularly, these diluents are not suitable for a water soluble type formulation. But addition of sugars as was done may pose problem of cake type mass formation during storage due to the hygroscopic nature of these substances.

Recent article published in Indian Journal of Science and Technology, Vol 10(22), DO1:10.17485/ijst/2017/113598, June 2017, described that aqueous extract of neem seed kernel contains 670 ppm of Azadirachtin along with water soluble proteinous matter and carbohydrates. Spraydrying of this aqueous solution poses problem as the solid content is very low, coupled with hygroscopic nature of the solid thus obtained.

Disadvantages often associated with the prior art are: a) extraction using whole neem seeds bring in a lot of impurities, thereby the purity of azadiractin remains low, b) cross-contamination of polar and non-polar solvent streams, c) water accumulation in the polar solvent streams d) low azadiractin concentrations or yields in the extracted product e) addition of inert carriers either brings acidity/alkalinity problem or cake type of mass formation during storage, and f) hygroscopic nature of the spray dried aqueous extract of neem seed kernel.

### BRIEF SUMMARY OF THE INVENTION

The presently disclosed embodiments overcome, diminish or eliminate the problems of the prior art by presenting methods and systems in extracting with water alone and then adding 6% azadirachtin containing SoluNeem for stability and efficacy of the formulation. The coextraction of azadiractin with carbohydrates and proteins has been taken as advantage to further enrich the aqueous solution with more of azadiractin. Though azadiractin solubility is very negligible in water (0.26 gms/l of 40-45% containing azadiractin A and B i.e 260 ppm), clarified aqueous extract contains anywhere between 300 and 800 ppm. This indicated to us that the presence of water soluble carbohydrates and proteins could be helpful in increasing the solubility of azadiractin. Based on this observation we probed the maximum solubility of azadiractin in aqueous extract. U.S. Pat. No. 6,635,757 describes the preparation of water soluble SoluNeem by complexation of all active ingredients of neem seed kernel extract containing a minimum of 32% azadiractin with Beta Methyl Cyclodextrin (BMCD). It is known that cyclodextrins are able to encapsulate various organic guests within their hydrophobic cavities to afford host-guest complex or supramolecular species in aqueous solution. Hence it is quite understandable that carbohydrates/proteins present in neem seeds can encapsulate azadiractin in their hydrophobic cavities making more azadiractin soluble in water. Further this process is particularly advantageous as the aqueous extract is spray dried which completely eliminates generation of waste water which otherwise cause pollution problem of biological oxygen demand (BOD). Also this water soluble formulation contains azadirachtin of more than 6% along with native water soluble ingredients of neem seed kernel.

### BRIEF DESCRIPTION OF THE DRAWINGS

FIG. 1 is table of findings

These and other aspects of the present invention will become apparent upon reading the following detailed description in conjunction with the associated drawings.

### DETAILED DESCRIPTION OF EMBODIMENTS OF THE INVENTION

The following detailed description is directed to certain specific embodiments of the invention. However, the invention can be embodied in a multitude of different ways as defined and covered by the claims and their equivalents. In this description, reference is made to the drawings wherein like parts are designated with like numerals throughout.

Unless otherwise noted in this specification or in the claims, all of the terms used in the specification and the claims will have the meanings normally ascribed to these terms by workers in the art.

Unless the context clearly requires otherwise, throughout the description and the claims, the words “comprise,” “comprising” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “above,” “below,” and words of similar import, when used in this application, shall refer to this application as a whole and not to any particular portions of this application.

A first aspect of a disclosed embodiment may comprise a process for the production of water soluble azadirachtin and its enrichment both by concentrating the aqueous extract and by addition of external azadiractin and finally making a water soluble powder that may contain inclusion complexes, preferably a mixture of cyclodextrin derivatives and polysaccharide-proteins, especially for such production at commercial scale:

The neem seeds are preferably first dried to a moisture content of from 10-15% by weight to a level of 2-4%, and then the husk is removed to get the neem seed kernel. The neem seed kernel obtained is then pressed to produce the cake from which has been removed a substantial proportion of the oil content of the seeds. Preferably, the pressing is done at a low speed to reduce the temperature build up in the neem seeds during pressing and so minimize heat degradation of the azadirachtin. The pressing is preferably conducted so that at least 70% by weight, more preferably 90% by weight of the oil is removed from the neem seed kernels during the pressing process.

Generally, the extraction process of this invention is preferably accomplished using demineralised water as solvent, although one of skill would recognize that other polar solvents could be employed.

The aqueous solution obtained can be cooled and the oil is preferably separated from the water on the basis of the different densities of the oil and water. Preferably, the separation can be undertaken by passing the aqueous solution through a separator where the solution is spun through a series of cones that physically separate the oil and water on the basis of their densities. The oil stream which preferably contains no azadirachtin but does contain the less polar triterpenes and the diterpenes will be kept aside for oil recovery and further processing

The azadirachtin-rich aqueous solution, which also contains polar triterpenes, polysaccharides and proteins is passed through celite bed to get clear aqueous solution. The azadiractin content in the clear aqueous solution would generally vary from 300 to 1000 ppm depending upon neem seed quality and the proportion of water used for extraction and most preferably 8 volumes of water with respect to deoiled neem cake.

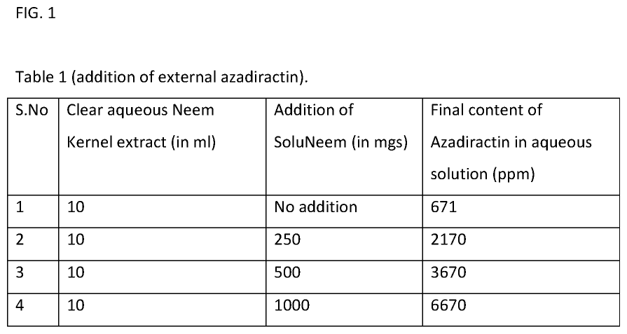

Another embodiment of this invention is enhancement of azadiractin content in the aqueous layer by concentrating it under vacuum to preferably ½ to ¼ volume and then by the addition of external azadiractin powder viz., SoluNeem, stirring and filtration. This way the azadiractin content in the aqueous layer could be enhanced by many fold, preferably having 600 ppm and above. These findings are illustrated in FIG. 1, showing Table 1

In a still further embodiment, the process can include addition of inclusion complex preferably a mixture of beta-cyclodextrin, beta-methyl cyclodextrins along with polysaccharide-protein complex with limonoids present in neem seed kernels preferably so as to get azadiractin enriched aqueous solution and finally spray drying to get enriched water soluble azadiractin containing powder.

The invention provides for an efficient process for producing water soluble azadirachtin containing powder formulation from neem seed kernel with the help of native and external inclusion complexes. This process comprises expelling a large proportion of oil from neem seed kernels (NSK) by cold pressing, extracting the so called de-oiled neem seed cake with water to form an aqueous solution in which the azadirachtin is complexed with water soluble polysaccharides and proteins. The enrichment of azadiractin in aqueous solution can be achieved by addition of azadiractin containing inclusion complex and spray drying. This helps in retaining all naturally occurring limonoids of neem seed kernel along with native encapsulating agents such as polysaccharide-protein matrix and also achieves water soluble powder with higher amounts of azadirachtin (>6000-60,000 ppm) in it.

Specifically, in its most preferred embodiment, the invention discloses a water soluble powder formulation comprising at least one or two inclusion complexes and a neem seed kernel extract, wherein said neem seed kernel extract comprises azadirachtin, limonoids and polysaccharide-protein matrix, wherein said composition is a free flowing powder, and wherein said inclusion complexes and said neem seed kernel extract are not chemically bonded. The said inclusion compound is preferably a cyclodextrin derivative and azadirachtin is preferably present in an amount exceeding 600 ppm. The azadirachtin in the formulation is an azadirachtin A or an azadirachtin B, or both.

Another preferred inclusion complex is cyclodextrin derivative and polysaccharide-protein mixture thereof.

The invention also provides for a process for the production of water soluble azadirachtin enriched water soluble powder. The process steps include:

(i) Removal of husk from the neem seeds to get neem seed kernel

(ii) de-oiling by cold pressing neem seeds kernel to remove a first proportion of the oil content thereof

(iii) extracting the so called de-oiled neem seeds cake with water at near neutral pH

(iv) separating the liquid phase to obtain clarified aqueous solution of azadirachtin along with polysaccharides and proteins and the remaining oil.

Further, optional step comprise:

(v) concentration of clarified aqueous extract to suitable extent under vacuum so that azadiractin content would preferably be nearly doubled

(vi) addition of external azadirachtin containing SoluNeem® in different ratios to the clarified aqueous extract so that azadiractin content would be increased many fold

(vii) filteration to remove the water insolubles

(ix) spray drying the resultant solution from step vii to obtain enriched water soluble azadiractin powder.

### Examples 1

Neem seeds, 500 kgs, were dehulled and deoiled by cold pressing using standard methodology involving mechanical oil extrusion. 100 kgs of deoiled neem cake was ground and stirred with 800 kg of demineralised water in Stainless steel reactor at room temperature for 5 hours. The slurry was filtered to remove majority of solids. The aqueous layer was finally filtered using Nutsch filter with celite bed to get clear solution of 730 kgs. This clear aqueous solution contained 671 ppm of azadiractin. To this was added different quantities of SoluNeem powder and stirred at room temperature for 4 hours and filtered to remove insolubles. The results are tabulated in Table 1.

### Example 2

To 10 lts of aqueous extract of neem seed kernel when added 60 gms of SoluNeem and filtered. The clear aqueous solution should contain about 6670 ppm of azadiractin.

### Example 3

To 10 lts of aqueous extract of neem seed kernel when added 30 gms of SoluNeem and filtered. The clear aqueous solution should contain about 3670 ppm of azadiractin.

### Example 4

To 10 lts of aqueous extract of neem seed kernel when added 15 gms of SoluNeem and filtered. The clear aqueous solution should contain about 2170 ppm of azadiractin.

Various embodiments are possible of the invention other than those disclosed above and are easily comprehended by a person skilled in the art. The invention encompasses all such embodiments within its scope.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not only the systems described herein. The various embodiments described herein can be combined to provide further embodiments. These and other changes can be made to the invention in light of the detailed description.

All the above references and U.S. patents and applications are incorporated herein by reference. Aspects of the invention can be modified, if necessary, to employ the systems, functions and concepts of the various patents and applications described above to provide yet further embodiments of the invention.

These and other changes can be made to the invention in light of the above detailed description. In general, the terms used in the following claims, should not be construed to limit the invention to the specific embodiments disclosed in the specification, unless the above detailed description explicitly defines such terms. Accordingly, the actual scope of the invention encompasses the disclosed embodiments and all equivalent ways of practicing or implementing the invention under the claims.

While certain aspects of the invention are presented below in certain claim forms, the inventors contemplate the various aspects of the invention in any number of claim forms.

What is claimed is:

1. A water soluble powder formulation comprising one or more inclusion complexes and a neem seed kernel extract, wherein said neem seed kernel extract comprises azadirachtin, limonoids and polysaccharide-protein matrix, wherein said formulation is a free flowing powder, and wherein said inclusion complexes and said neem seed kernel extract are not chemically bonded.

2. The water soluble powder formulation of claim 1, wherein said inclusion complex comprises a cyclodextrin derivative.

3. The water soluble powder formulation of claim 1, wherein said azadirachtin is present in an amount exceeding 600 ppm.

4. The water soluble powder formulation of claim 1, wherein said azadirachtin is selected from the group comprising: azadirachtin A, or azadirachtin B, or mixtures thereof.

5. The water soluble powder formulation of claim 1, wherein said inclusion complex comprises a cyclodextrin derivative and a polysaccharide-protein mixture.

6. A process for the production of water soluble azadirachtin enriched water soluble powder, the process comprising:
(i) removal of husk from neem seeds to obtain neem seeds kernel,
(ii) de-oiling by cold pressing neem seeds kernel to remove a first proportion of an oil content thereof, resulting in a de-oiled neem seeds cake,
(iii) extracting the cake from the de-oiled neem seeds with water at near neutral pH
(iv) separating a liquid phase to obtain clarified aqueous solution of azadirachtin along with polysaccharides and proteins and remaining oil.

7. The process of claim 6, for the production azadirachtin enriched water soluble powder further comprising:
(v) concentration of clarified aqueous extract to suitable extent under vacuum so that azadiractin content is nearly doubled;
(vi) addition of external azadirachtin containing SoluNeem® in different ratios to the clarified aqueous extract so that azadiractin content is increased;
(vii) filteration to remove the water insoluble;
(ix) spray drying the resultant solution from step vii to obtain enriched water soluble azadiractin powder.

8. The process of claim 6 wherein the cake is extracted with heated water or steam.
